(19) United States
(12) Patent Application Publication
(10) Pub. No.: US 2011/0166077 A9
(43) Pub. Date: Jul. 7, 2011

(54) COMBINED THERAPY AGAINST TUMORS COMPRISING SUBSTITUTED ACRYLOYL DISTAMYCIN DERIVATIVES AND RADIOTHERAPY

(75) Inventors: Maria Cristina Rosa Geroni, Milano (IT); Jennifer Margaret Tursi, Milano (IT); Camilla Fowst, Milano (IT)

(73) Assignee: Pharmacia Italia S.p.A., Milan (IT)

(21) Appl. No.: 10/957,507

(22) Filed: Oct. 1, 2004

Related U.S. Application Data
(63) Continuation of application No. PCT/EP03/03192, filed on Mar. 17, 2003.

(30) Foreign Application Priority Data
Apr. 2, 2002 (EP) 02076240.7

(51) Int. Cl. A61K 38/06; A61K 31/4025

(52) U.S. Cl. 514/19.3; 514/422; 514/21.9; 424/648; 514/19.4

(57) ABSTRACT

The present invention provides the use of acryloyl distamycin derivatives, in particular α-bromo- or α-chloro-acryloyl distamycin derivatives, in combination with radiotherapy, for the treatment of tumors.

### CROSS REFERENCE TO RELATED APPLICATIONS

This application is based upon and claims the benefit of priority from European Patent Application No. 02076240.7 filed Apr. 2, 2002, the entire contents which is expressly incorporated herein by its reference.

### FIELD OF THE INVENTION

The present invention relates to the field of cancer treatment and provides an antitumor therapy comprising the combined use of a substituted acryloyl distamycin derivative, more particularly a α-bromo- or α-chloro-acryloyl-distamycin derivative, with radiotherapy.

### BACKGROUND OF THE INVENTION

The treatment of tumours with ionizing radiation, also referred to as radiotherapy, is extensively used in cancer therapy as it provides destruction of tumour cells together with inhibition of tumour cell growth, presumably through DNA damage.

Some therapeutic compounds, which are known as being cytotoxic per se, hence susceptible of being used in the therapy of cancer, are also endowed with radiosensitisation activity as they are capable of inducing DNA radiation damage in response to ionizing radiation.

So far, the possibility of combining both cytotoxic agents, e.g. a given radiosensitiser and radiotherapy, with the expectation of getting a supra-additive antitumor effect in comparison to the single cytotoxics alone, is of utmost importance in cancer therapy.

Among the several compounds endowed with antitumor activity and also known as possessing radiosensitisation activity see, for instance, cisplatin, gemcitabine, navelbine, tomudex, nicotinamide, paclitaxel, docetaxel, simvastatin and topotecan.

In addition, the use of halogenated DNA ligands as possible radiosensitisers, also including some distamycin derivatives, were disclosed by R. Martin et al. in the international patent application WO 90/12321.

For a general reference to distamycin, an antibiotic substance with antiviral and antiprotozoal activity, as well as to the several derivatives thereof which are known as cytotoxic agents see, for instance, Nature 203:1064 (1964); J. Med. Chem. 32:774-778 (1989); and the international patent application WO 90/11277, WO 98/21202, WO 99/50265, WO 99/50266 and WO 01/40181, all in the name of the applicant itself and herewith incorporated by reference.

Among the several distamycin derivatives being disclosed so far, a class of α-bromo- or α-chloro-acryloyl-distamycins, as per the aforementioned international patent application WO 98/04524, were found to possess a significant antineoplastic activity.

The present inventors have now found that these same compounds are also unexpectedly endowed with a remarkable radiosensitisation activity which render their use, in combination with radiotherapy, particularly advantageous in cancer therapy.

### SUMMARY OF THE INVENTION

It is therefore a first object of the present invention, the use of a α-bromo- or α-chloro-acryloyl-distamycin derivative in the preparation of a medicament having radiosensitisation activity.

### DETAILED DESCRIPTION OF THE INVENTION

In the present description, unless otherwise specified, with the term “radiosensitisation activity” it is intended the aforementioned capability of a compound, or medicament thereof, to act as a radiosensitiser. With the term “radiosensitiser”, in its turn, refers to a compound or medicament which is capable of increasing or otherwise improving tumor cells destruction in response to ionizing radiation.

Finally, the term “ionizing radiation” is the one conventionally adopted in the therapeutic field of cancer treatment and includes photons having enough energy for bonds ionization such as, for instance, α-, β- and γ-rays from radioactive nuclei as well as x-rays.

According to a preferred aspect of the invention, the α-bromo- or α-chloro-acryloyl-distamycin-derivative is a compound of formula (I) below 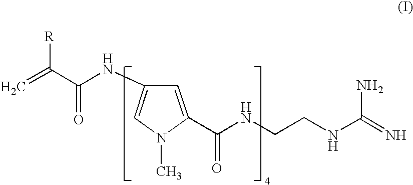
wherein R is a bromine or chlorine atom, more preferably bromine, or a pharmaceutically acceptable salt thereof.

Pharmaceutically acceptable salts of the compounds of formula (I) are the salts with pharmaceutically acceptable inorganic or organic acids such as, for instance, hydrochloric, hydrobromic, sulfuric, nitric, acetic, propionic, succinic, malonic, citric, tartaric, methanesulfonic, p-toluenesulfonic acid and the like; the hydrochloride salt being the preferred one.

Even more preferably, the acryloyl-distamycin derivative for use as radiosensitiser is the compound N-[5-[[[5-[[[2-[(animoiminomethyl)amino]ethyl]amino]carbonyl]-1-methyl-1H-pyrrol-3-yl]amino]carbonyl]-1-methyl-1H-pyrrol-3-yl]-4-[[[4-[(2-bromo-1-oxo-2-propenyl)amino]-1-methyl-1H-pyrrol-2-yl]carbonyl]amino]-1-methyl-1H-pyrrole-2-carboxamide hydrochloride (Compound A).

The combined therapy of the invention is suitable for the treatment of various tumor forms such as, for instance, breast, ovary, lung, colon (including rectus), kidney, stomach, pancreas, liver, head and neck, esophagus, uterus (including body and cervix), vagina, melanoma and non-melanoma skin cancer, as well as sarcomas.

From all the above and unless otherwise specified, it is clear to the skilled person that the α-bromo- or α-chloro-acryloyl-distamycin derivative may be administered to mammals, including humans, through the usual routes, for example parenterally, e.g. by intravenous injection or infusion.

The dosage will depend from several factors, also including the selected schedule of administration which may comprise repeated doses, for instance once a day, once a week, twice a week, and the like, as the case may be.

As a non limiting example, suitable dosages may range from about 0.05 mg/m² to about 10 mg/m².

For any indication concerning suitable pharmaceutical forms for administering the acryloyl-distamycin derivatives in re, hence including any pharmaceutically acceptable excipient, see the aforementioned international patent application WO 98/04524.

A further aspect of the present invention is to provide a method of treating a mammal, including humans, suffering from a neoplastic disease state, which method comprises administering to said mammal a α-bromo- or α-chloro-acryloyl-distamycin derivative and radiotherapy, in amounts and according to a schedule treatment effective to produce a synergistic antineoplastic effect.

By the term “synergistic” effect, as used herein, it is meant the inhibition of the growth tumor, preferably the complete regression of the tumor, by administering an effective amount of the above acryloyl distamycin derivative and the ionizing radiation to mammals, including humans. By the term “administered” or “administering”, as used herein, it is meant parenteral (e.g. intravenous) administration.

As far as the schedule treatment is concerned, exposure to radiotherapy may either occur simultaneously whilst administering the medicament comprising the α-bromo- or α-chloro-acryloyl-distamycin derivative or, alternatively, sequentially in any order.

Preferably, the schedule treatment first comprises administering the drug to the patient which only subsequently is subjected to radiotherapy exposure.

According to the present invention, the acryloyl distamycin derivative may be also administered with additional antitumor agents such as, for instance, topoisomerase I or II inhibitors, e.g. CPT-11, topotecan, 9-amino-camptothecin, 9-nitro-camptothecin, 10,11-methylenedioxy-camptothecin, doxorubicin, daunorubicin, epirubicin, nemorubicin, idarubicin, etoposide, teniposide, mitoxanthrone, losoxantrone, amsacrine, actinomycin D; alkylating agents, e.g. melphalan, chlorambucil, mechlorethamine, cyclophosphamide, ifosfamide, busulfan, carmustine, lormustine, semustine, fotemustine, decarbazine, temozolide, thitepa, mitomycin C, cisplatin, carboplatin, oxaliplatin, nedaplatin, lobaplatin; antimicrotubule agents, e.g. paclitaxel, docetaxel, vincristine, vinblastine, vindesine, vinorelbine, estramustine; antimetabolites, e.g. metotrexate, trimetrexate, tomudex, 5-FU, floxuridine, ftorafur, capecitabine, cytarabine, azacitidine, gemcitabine; protein kinase inhibitors, e.g. STI571 (Gleevec), ZD-1839 (Iressa), OSI-774 (Tarceva), SU 5416 (Semaxanib), SU 6668, SU 11248; retinoid derivatives, e.g. cis-retinoic acids, trans-retinoic acids; cyclooxygenase inhibitors such as COX-2 inhibitors, e.g. celecoxib, rofecoxib, parecoxib, valdecoxib; hormonal agents, e.g. exemestane, formestane, atamestane, letrozole, fadrozole, anastrozole.

According to a preferred embodiment of the invention, the use of a α-bromo- or α-chloro-acryloyl-distamycin derivative with radiotherapy also comprises the administration of a platinum alkylating agent, more preferably cisplatin.

### Pharmacology

The remarkable radiosensitisation effect exerted by the α-bromo- or α-chloro-acryloyl-distamycin derivatives, in particular the compounds of formula (I), is shown according to in vitro clonogenic assays on SQ20B (radiation-resistant human squamous cell carcinoma of the larynx) and A431 (human vulval carcinoma) cell lines. In this respect, two different schedule treatments were evaluated either comprising simultaneous exposure to the tested compound of formula (I) and to radiation, or sequential exposure to both these cytotoxic agents in any order, that is drug/radiation or radiation/drug (see details below). As control, the effect of cisplatin in combination with radiotherapy has been tested in the same operative conditions.

To define a Sensitization Ratio (SR), the clonogenic survival of cells being treated with a combination of irradiation and drug exposure (S_{X+D}) was compared with the product of survival for drug alone (S_{D}) and irradiation alone (S_{X}), as follows
SR=S_{X+D}/S_{D}*S_{X}

From the above, it is clear to the skilled person that if both radiation and drug exerted their cytotoxic effect independently from each other, SR values would be close to 1 whereas, on the contrary, a radiosensitisation effect indicating a synergism between ionizing radiation and drug is characterized by SR values lower than 1 (SR<1).

Analysis of the obtained results in any of the experiments being carried out clearly indicate that the tested compound of formula (I) exerts a remarkable and statistically significant radiosensitising effect.

In particular, whilst sensitization is substantially comparable to that of cisplatin on SQ20B cell line, it is unexpectedly and significantly superior than that of cisplatin on A431 cell line, hence indicating a possible widest range of applications for the compounds of formula (I), in combination with radiotherapy.

In addition to the above, it has been unexpectedly found in accordance with the present invention that the radiosensitisation effect of the compound of formula (I) could be even increased, to a statistically significant extent, when drug exposure occurred before irradiation treatment, according to one of the sequential schedule treatments.

To better illustrate the present invention, without posing any limitation to it, the following examples are now given.

### EXAMPLE 1

Radiosensitisation Activity of Compound A in Comparison to Cisplatin

For both compounds Compound A and cisplatin, exposures were simultaneous to ionizing radiation in both SQ20B and A431 cell lines. The schedule consisted of 2 h drug treatment with a period of irradiation (10 minutes) starting at the beginning of the 2^{nd} hour of treatment.

Four data sets for each of Compound A and cisplatin, in each cell line were obtained (see table 1) comprising duplicates of two different drug concentration chosen to yield cytotoxicity values corresponding to 80% (C₈₀) and 20% (C₂₀) survival for treatment with the drug alone.

TABLE 1
Sensitisation ratio of Compound A and cisplatin in combination with
radiotherapy

| | | | Sensitization Ratio SR^{(b)} | |
|---|---|---|---|---|
| | | Drug | Values for | Mean of |
| Cell line | Drug | Concentration^{(a)} | each culture | duplicates |
| SQ20B | Compound A | 50 (C₈₀)^{(c)} | 0.85 | C₈₀ 0.85 |
| | | 350 (C₂₀)^{(d)} | 0.46 | C₂₀ 0.50 |
| | | 50 (C₈₀) | 0.84 | p = 0.017 |
| | | 350 (C₂₀) | 0.55 | |
| | cisplatin | 0.4 (C₈₀) | 0.78 | C₈₀ 0.80 |
| | | 6.5 (C₂₀) | 0.72 | C₂₀ 0.77 |
| | | 0.4 (C₈₀) | 0.82 | p = 0.034 |
| | | 6.5 (C₂₀) | 0.81 | |
| A431 | Compound A | 20 (C₈₀) | 0.65 | C₈₀ 0.62 |
| | | 90 (C₂₀) | 0.33 | C₂₀ 0.37 |
| | | 20 (C₈₀) | 0.59 | p = 0.029 |
| | | 90 (C₂₀) | 0.40 | |
| | cisplatin | 0.8 (C₈₀) | 1.02 | C₈₀ 0.96 |
| | | 5.0 (C₂₀) | 1.08 | C₂₀ 0.97 |
| | | 0.8 (C₈₀) | 0.84 | p = 0.37 |
| | | 5.0 (C₂₀) | 0.86 | |

^{(a)}Expressed as ng/ml for Compound A and μM for cisplatin;
^{(b)}SR values lower than 1 (SR < 1) indicate radiosensitisation;
^{(c)}C₈₀ drug concentration corresponding to 80% cell survival
^{(d)}C₂₀ drug concentration corresponding to 20% cell survival

From the above, SR for Compound A is lower than 1 in both cell lines being investigated; on A431, SR for Compound A is markedly lower than that of cisplatin, hence indicating a superior radiosensitisation effect.

### EXAMPLE 2

Radiosensitisation Activity of Compound A Under Sequential Schedule Treatment

Compound A was tested in both SQ20B and A431 cell lines, according to two sequential schedule treatments comprising: 2 h drug treatment ending 60 minutes before irradiation (drug-before schedule) and 2 h drug treatment starting 40 minutes after irradiation (drug-after schedule), the irradiation period being of 10 minutes, in each case. For each cell line, Compound A was tested at the highest concentration (see table 2) to yield cytotoxicity values corresponding to 20% (C₂₀) survival for treatment with the drug alone.

TABLE 2
Effect of the sequence of treatment on the sensitization ratio of Compound
A in combination with radiotherapy

| | |
|---|---|
| Drug | |
| Concentration | Sensitization Ratio SR^{(a)} |

| Cell line | (ng/ml) | Drug-before^{(b)} | Drug-after^{(c)} |
|---|---|---|---|
| SQ20B | 350 (C₂₀)^{(d)} | 0.15 | 0.62 |
| | 350 (C₂₀) | 0.47 | 0.73 |
| A431 | 90 (C₂₀) | 0.11 | 0.87 |
| | 90 (C₂₀) | 0.13 | 0.43 |

| | | |
|---|---|---|
| Paired t-test | P = 0.043 | P = 0.074 |

^{(a)}SR values lower than 1 (SR < 1) indicate radiosensitisation;
^{(b)}2 h exposure to Compound A before irradiation;
^{(c)}2 h exposure to Compound A after irradiation;
^{(d)}C₂₀ drug concentration corresponding to 20% cell survival

From the above, even if SR values are lower than 1 in both cell lines and according to both schedules, the radiosensitisation activity of Compound A is significantly higher (SR<1) when the treatment with the compound is carried out before irradiation.

1. Use of a α-bromo- or α-chloro-acryloyl-distamycin derivative in the preparation of a medicament having radiosensitisation activity.

2. Use according to claim 1 wherein the α-bromo- or α-chloro-acryloyl-distamycin derivative is of formula (I) 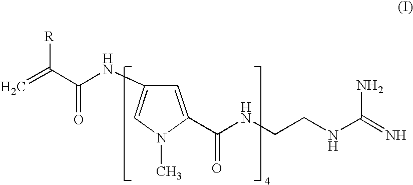
wherein R is a bromine or chlorine atom, or a pharmaceutically acceptable salt thereof.

3. Use according to claim 2 wherein, with formula (I), R is a bromine atom.

4. Use according to claim 1 wherein the acryloyl-distamycin derivative is the compound N-[5-[[[5-[[[2-[(aminoiminomethyl)amino]ethyl]amino]carbonyl]-1-methyl-1H-pyrrol-3-yl]amino]carbonyl]-1-methyl-1H-pyrrol-3-yl]-4-[[[4-[(2-bromo-1-oxo-2-propenyl)amino]-1-methyl-1H-pyrrol-2-yl]carbonyl]amino]-1-methyl-1H-pyrrole-2-carboxamide hydrochloride.

5. Use according to claim 1 for the treatment of tumors selected from the group consisting of breast, ovary, lung, colon (including rectus), kidney, stomach, pancreas, liver, head and neck, esophagus, uterus (including body and cervix), vagina, melanoma and non malanoma skin cancer, as well as sarcomas.

6. A method of treating a mammal, including humans, suffering from a neoplastic disease state, which method comprises administering to said mammal α-bromo- or α-chloro-acryloyl-distamycin derivative in combination with radiotherapy, in amounts and according to a schedule treatment effective to produce a synergistic antineoplastic effect.

7. The method of claim 6 wherein the α-bromo- or α-chloro-acryloyl-distamycin derivative is of formula (I) 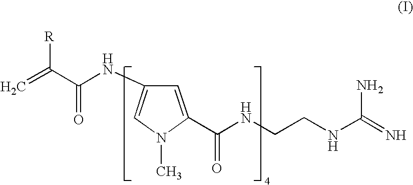
wherein R is a bromine or chlorine atom, or a pharmaceutically acceptable salt thereof.

8. The method of claim 7 wherein, within formula (I), R is a bromine atom.

9. The method of claim 6 wherein the acryloyl-distamycin derivative is the compound N-[5-[[[5-[[[2-[(aminoiminomethyl)amino]ethyl]amino]carbonyl]-1-methyl-1H-pyrrol-3-yl]amino]carbonyl]-1-methyl-1H-pyrrol-3-yl]-4-[[[4-[(2-bromo-1-oxo-2-propenyl)amino]-1-methyl-1H-pyrrol-2-yl]carbonyl]amino]-1-methyl-1H-pyrrole-2-carboxamide hydrochloride.

10. The method of claim 6 wherein the neoplastic disease state is selected from the group consisting of breast, ovary, lung, colon, kidney, stomach, pancreas, liver, head and neck, esophagus, uterus, vagina, melanoma and non malanoma skin cancer, as well as sarcomas.

11. The method of claim 6 wherein exposure to radiotherapy occurs either simultaneously whilst administering the medicament of claim 1, or sequentially, in any order.

12. The method of claim 6 first comprising administering the α-bromo- or α-chloro-acryloyl-distamycin derivative to the patient and subsequently subjecting the said patient to radiotherapy.

13. The method according to claim 6 optionally further comprising the administration of an additional antitumor agent, either separately, simultaneously or sequentially, in any order.

14. The method of claim 13 wherein the additional antitumor agent is selected from the group consisting of topoisomerase I or II inhibitors, alkylating agents, antimicrotubule agents, antimetabolites, protein kinase inhibitors, retinoid derivatives, cyclooxygenase inhibitors and hormonal agents.

15. The method of claim 14 wherein the additional antitumor agent is cisplatin.

16. A pharmaceutical composition comprising a α-bromo or α-chloro-acryloyl-distamycin derivative of formula (I) or pharmaceutically acceptable salt thereof, as defined in claim 2, for use as a radiosensitizer.

17. The pharmaceutical composition of claim 16 wherein the derivative of formula (I) is as defined in claim 4.
